(19) United States
(12) Patent Application Publication
(10) Pub. No.: US 2012/0027715 A1
(43) Pub. Date: Feb. 2, 2012

(54) POLYMER FILMS WITH EMBEDDED IODINATED RESIN AND METHODS OF MANUFACTURING SAME

(75) Inventors: Pierre J. Messier, Quebec (CA); David Ohayon, (US)

(73) Assignee: TRIOMED INNOVATIONS CORP., Williston, VT (US)

(21) Appl. No.: 13/195,609

(22) Filed: Aug. 1, 2011

Related U.S. Application Data
(60) Provisional application No. 61/370,034, filed on Aug. 2, 2010.

(51) Int. Cl. A01N 27/00; A61P 31/00; A61P 17/02; A01N 37/18; A01P 1/00

(52) U.S. Cl. 424/78.31; 424/78.37

(57) ABSTRACT

The disclosure relates to polymer films containing iodinated resin, where such films have advantageous antibacterial and/or antitoxic properties. The films may be used, for example, in medical tape, wound dressings and bandages. In certain embodiments, the invention is also directed to methods of manufacturing such polymer films, where various manufacturing difficulties have been addressed and overcome.

### CROSS-REFERENCE TO RELATED APPLICATION

This application claims priority to and the benefit of, and incorporates herein by reference in its entirety, U.S. provisional Patent Application No. 61/370,034 which was filed on Aug. 2, 2010.

### FIELD OF INVENTION

This invention relates generally to polymer films and their methods of manufacture. More particularly, in certain embodiments, the invention relates to polymer films containing iodinated resin and having advantageous antitoxic properties.

### BACKGROUND OF INVENTION

With the increasing demand for antimicrobial products in the healthcare, nutritional, packaging, and clothing industries, new technologies continue to emerge with the intent of increasing efficacy and minimizing safety concerns associated with such products. Thin films containing an antimicrobial agent have generated attention in recent years, particularly in the healthcare industry. Incorporation of an antimicrobial agent into a film applied to the surface of an object may provide a means of reducing the total amount of the antimicrobial agent necessary to retain high levels of efficacy. Such antimicrobial films could potentially be useful in hospital settings and wound management. The majority of antimicrobial wound dressings currently rely on incorporating the active agent into the wound dressing itself, such as in a nonwoven or gauze. Other techniques rely on incorporating the active agent into an adhesive which is applied directly on the skin. Besides being costly, the current wound management techniques usually suffer from less than ideal efficacy. To compensate, the active agent is applied in excess concentration, which leads to toxicity related problems. The application of a thin film in wound management is intended to mitigate these problems.

Thin films incorporating such active agents as silver, zinc and Schiff bases are currently being developed for wound care management. While some success has been achieved, the films have not been able to provide the desired levels of efficacy and safety.

More potent antimicrobial agents such as iodine have not been effectively incorporated into films for wound management or other applications. Although it is well-known that iodine is a broad spectrum antimicrobial agent, uncontrolled release of iodine is associated with toxicity. In the field of wound management, the toxicity associated with iodine has been controlled to a certain degree through incorporation of iodine into nonwoven wound dressings and in adhesives applied directly to the skin. However, such wound dressings do not provide maximum efficacy and do not provide maximum ‘kill’ rates. In nonwovens, for instance, iodine must travel from within the fibers or the interstitial matrix of the fibers to the surface of the object in order to come in contact with a microbe. A thin film, on the other hand, could potentially provide a high concentration of iodine on its surface, thereby increasing efficacy and increasing the ‘kill’ rate. However, techniques for adding liquid iodine or solid iodine to a polymeric mixture for the purposes of thin film formation have not been effectively developed. For instance, techniques to compound iodine with a polymer followed by extrusion have failed due, in part, to the negative reaction of pure iodine on the parts of the compounder or extruder.

Accordingly, a nontoxic thin film containing a broad-spectrum antimicrobial agent such as iodine would have considerable use in wound management and other applications.

### SUMMARY OF INVENTION

The disclosure relates to polymeric films containing iodinated resin, where such films have advantageous antitoxic properties, including antimicrobial, antifungal and antiviral properties. The antitoxic films of the present invention are capable of exerting a toxic effect on a diverse array of microorganisms and other pathogens and environmental toxins without being harmful to humans. The films may be used, for example, in wound dressings and bandages. The films may also be used, for example, as (or in) packaging material; food wrap; house wrap (e.g., air/water/moisture barriers used in construction); automobile, boat, and camper covers (and other surface covers); industrial packaging; medical packaging; sports apparel and protective apparel.

In one aspect, the disclosure relates to a thin polymer film made with polymer and iodinated resin. In certain embodiments, the polymer comprises polypropylene (PP), polyethylene (PE), nylon, ethylene-methyl acrylate copolymer (EMAC), and/or polyvinyl chloride (PVC). In certain embodiments, the iodinated resin is an iodinated anionic resin such as Triosyn®, and is integrated into the polymer film.

In another aspect, the disclosure is directed to a thin polymer film comprising a polymer and an iodinated resin powder. In certain embodiments, the surface area of the iodinated resin powder is in the range of from about 1.0 m²/g to about 3.0 m²/g.

In yet another aspect, the disclosure relates to a medical and/or surgical tape comprising a polymeric thin film containing a plurality of iodinated resin particulates. In certain embodiments, the surface area of the iodinated resin particulates is in the range of from about 1.0 m²/g to about 3.0 m²/g.

In still another aspect, the disclosure relates to a wound management film comprising a polymer selected from the group consisting of polypropylene (PP), polyethylene (PE), nylon, hydrocolloid, ethylene-methyl acrylate copolymer (EMAC), and polyvinyl chloride (PVC) and an iodinated resin, wherein said film releases iodine in a controlled manner upon contact with the skin. In certain embodiments, the film is occlusive. In other embodiments, the film is non-occlusive.

The disclosure is also directed to methods and systems for manufacturing such films. Thus, in another aspect, the invention is directed to a method for manufacturing a polymer film integrated with iodinated resin, the method comprising the steps of: introducing an iodinated resin powder into an extruder, introducing polymer into the extruder, operating the extruder at a temperature such that the polymer is molten, cooling the extrudate to at least partially solidify the extrudate, cutting the extrudate into small pieces (e.g., pelletize the extrudate), sandwiching the small pieces of extrudate between two sheets of Teflon, compressing the sheets at high temperature and pressure, removing the polymer film formed between the sheets, and cooling the film. In certain embodiments, the polymer/iodinated resin extrudate are cut into pellets having an average height of about 5 mm to about 9 mm (e.g., about 7 mm), and an average diameter/length of about 2 mm to about 5 mm (e.g., about 3 to about 4 mm). In certain embodiments, the polymer film comprises polypropylene (PP), polyethylene (PE), nylon, ethylene-methyl acrylate copolymer (EMAC), and/or polyvinyl chloride (PVC). In certain embodiments, the extruder is a twin screw extruder. In preferred embodiments, the extruder is coated and/or made of a hasteloy metal to improve resistance to corrosion due to contact with the iodinated resin. In certain embodiments, the extruder has a liner to improve its corrosion resistance.

### BRIEF DESCRIPTION OF THE FIGURES

FIG. 1 graphically illustrates the efficacy of polypropylene films containing iodinated resin powder produced in accordance with the present disclosure against the challenge organism Pseudomonas aeruginosa in a time period ranging between 5 minutes and 24 hours.

FIG. 2 graphically illustrates the efficacy of polyethylene films containing iodinated resin powder produced in accordance with the present disclosure against the challenge organism Pseudomonas aeruginosa in a time period ranging between 5 minutes and 24 hours.

FIG. 3 graphically illustrates the efficacy of nylon films containing iodinated resin powder produced in accordance with the present disclosure against the challenge organism Pseudomonas aeruginosa in a time period ranging between 5 minutes and 24 hours.

FIG. 4 graphically illustrates the efficacy of EMAC films containing iodinated resin powder produced in accordance with the present disclosure against the challenge organism Pseudomonas aeruginosa in a time period ranging between 5 minutes and 24 hours.

FIG. 5 graphically illustrates the efficacy of polyvinyl films containing iodinated resin powder produced in accordance with the present disclosure against the challenge organism Pseudomonas aeruginosa in a time period ranging between 5 minutes and 24 hours.

### DESCRIPTION OF THE INVENTION

The following sections describe exemplary embodiments of the present disclosure. It should be apparent to those skilled in the art that the described embodiments of the present disclosure provided herein are illustrative only and not limiting, having been presented by way of example only.

Throughout the description, where devices and systems are described as having, including, or comprising specific components, or where processes and methods are described as having, including, or comprising specific steps, it is contemplated that, additionally, there are devices and systems of the present invention that consist essentially of, or consist of, the recited components, and that there are processes and methods according to the present disclosure that consist essentially of, or consist of, the recited processing steps.

Embodiments may be performed as part of a continuous, semi-continuous, or batch process. It is contemplated that methods of the disclosure may be combined or supplemented with reactors, systems, or processes that are known in the art.

In a first aspect of the disclosure, polymer films made by integrating polymer and iodinated resin, where such films have advantageous antitoxic properties are described. The polymeric films are effective against a diverse array of microorganisms, including bacteria, fungi, and viruses. The polymer used in the polymer films may be, for example, polypropylene (PP), polyethylene (PE), nylon, ethylene-methyl acrylate copolymer (EMAC), hydrocolloid and/or polyvinyl chloride (PVC). The polymer may be chosen to provide desired strength, modulus, corrosion-resistance, moisture-resistance, and/or other physical or chemical properties of the desired end product that will be formed with (or of) the polymer film. In certain embodiments, the iodinated resin integrated into the polymer film is an iodinated anionic resin such as Triosyn®.

In particular embodiments, the iodinated resin is a demand disinfectant iodinated resin. Iodine/resin demand disinfectants are described, for example, in U.S. Pat. No. 5,639,452 (“the '452 patent”), to Messier, the entire contents of which are hereby incorporated by reference. The '452 patent describes a process for preparing an iodine demand disinfectant resin from an anion exchange resin. The demand disinfectant iodinated resins described in the '452 patent may be ground into a powder. One preferred demand disinfectant iodinated resin is Triosyn® brand iodinated resin powders made by Triomed Innovations (Canada) Inc., a division of TrioMed Innovations Corp of Vermont, USA. The particle sizes of the powders range from about 1 micron to about 150 microns. Preferably, the particle sizes should be in the range from about 4 microns to about 10 microns.

Triosyn® iodinated resin powders used in accordance with certain embodiments of the present invention are referred to as Triosyn® T-50 iodinated resin powder, Triosyn® T-45 iodinated resin powder, Triosyn® T-40 iodinated resin powder or Triosyn® T-35 iodinated resin powder. The base polymer used to manufacture such iodinated resins is Amberlite® (Rohm & Haas). These resins contain quaternary ammonium exchange groups with are bonded to styrene divinyl benzene polymer chains. Other base polymers could be used. The numbers refer to the approximate weight percentage of iodine relative to the resin. Powders with other weight percentages of iodine may also be used in accordance with the present invention. Different percentages of iodine in the iodinated resin powders will confer different properties to the powder, in particular, different levels of biocidal activity. The particular resin used is based on the desired application. Iodinated resin from other sources can also be used.

The disclosure also relates to unique methods and systems for integrating the iodinated resin with the polymer to produce polymer films. There are various challenges posed by the production of such films. For example, certain processing equipment parts in contact with iodinated resins may corrode easily, and exposing iodinated resin to too much heat may deactivate or reduce the effectiveness of the iodinated resin as a disinfectant, barrier, antimicrobial, and/or antifungal agent in the resulting polymer film. In the present disclosure, thin polymer films containing iodinated resin have been successfully manufactured, and experiments with such films are further described below.

The use of a twin screw extruder is preferred; however a single screw extruder may be used with a Zeon mixer if Triosyn® pellets are introduced into the extruder instead of Triosyn® powder. In one example, a 5-zone twin screw extruder is used, and the barrel, the screws, and the 5 zones are heated to approximately 350 degrees F. (where the polymer is PE) in order to maintain the polymer in a sufficiently molten state. A rule of thumb is that the extruder temperature should be approximately 25 to 50 degrees F. higher than the polymer melting point temperature. So, for example, use of ethylene-methyl acrylate copolymer (EMAC) would require an extruder temperature of about 230° F. and use of PE would require an extruder temperature of about 405° F. Where a given temperature is indicated herein, it is understood that a range of +/−10 degrees F. about the indicated temperature is also contemplated in alternative embodiments.

Triosyn® powder is added into the molten polymer downstream in any of the first 3 zones to ensure a good uniform mixture of the Triosyn® in the polymer. In certain embodiments, an amount of iodinated resin within a range of from about 5% w/w to about 50% w/w is used in the polymer film. In other embodiments, an amount of iodinated resin within a range of from about 10% w/w to about 40% w/w is used in the polymer film. In still other embodiments, an amount of iodinated resin within a range of from about 10% to about 30% w/w is used in the polymer film. In other embodiments the range of iodinated resin in the polymer film is from about 15% to about 30%, or from about 20% to about 30% iodinated resin w/w. In the above embodiments, the iodinated resin may have a weight percentage iodine relative to the resin, for example, of about 30%, about 35%, about 40%, about 45%, or about 50% w/w.

The percentage of iodinated resin that can be incorporated in the film is dependent upon the polymer being used. For example, thin films comprising up to 30% w/w of Triosyn® in PE can be prepared using procedures described in the present disclosure. Adding more than 30% Triosyn® was found to cause the polymer to remain in a semi liquid form, which prevented adequate incorporation of iodinated resin into the polymer film. When EMAC was used as the polymer, thin films comprising 40% w/w or more of Triosyn® were produced using methods described in the present disclosure.

It has been found that the efficacy of the iodinated resin films is also dependent upon surface area of the iodinated resin particulates in the polymer film. The greater the surface area of the particle in the film, the greater the interaction between the iodine and the microorganism. Triosyn® powders provide films with a much greater surface area of the iodinated resin than corresponding resin beads and fragments. Accordingly, a lower concentration of the iodinated resin is needed to achieve a desired level of efficacy at a sufficient rate. Triosyn® powders having a surface area in the range of 1.0 m²/g to about 3.0 m²/g are ideal for thin film applications described in the present disclosure, For instance, BET measurements using Triosyn® T-50 powder (4-10 micron) reveals that the powder has a surface area of approximately 2.0 m²/g to about 2.5 m²/g. As discussed below, the film achieves a high level of efficacy against a diverse array of microorganisms in a rapid time period.

Color or any other additive including other antimicrobial materials and/or wound healing enhancers (e.g., in the case of wound dressing) can be added, for example, via the otherwise unused extruder zones, in order to impart additional desired characteristics to the extruded polymer product. The extrudate comes out as a continuous line that falls into a water bath to cool off the molten mixture (polymer and Triosyn®). After cooling, the extrudate is cut into small components such as pellets for subsequent processing into a film. In certain embodiments, the polymer/iodinated resin extrudate are cut into pellets having an average height of about 5 mm to about 9 mm (e.g., about 7 mm), and an average diameter/length of about 2 mm to about 5 mm (e.g., about 3 to about 4 mm).

It is important to note that the parts of the twin screw extruder that come into contact with the iodinated resin should be coated or made from a corrosion resistant (e.g., a hasteloy metal) so that when put into contact with iodinated resin and heat, the parts will not start corroding. Corrosion would have the effect of shortening the lifespan of the manufacturing equipment and would prevent economically feasible production of the iodinated polymer film. A liner could be added that will protect the metal parts or could be coated with an additive that will avoid corrosion over the long haul.

In the final preparation step, the cooled pellets comprising polymer and iodinated resin are made into a film. It is preferable to preheat for about 15 minutes the apparatus that will be used to make the film from the cooled polymer/iodinated resin pellets. In one embodiment, the apparatus has two metal plates that are heated to about 350° F. Once the apparatus reaches its target temperature for the polymer, an amount of about 1.0 gram of compounded polymer and iodinated resin is sandwiched between two Teflon sheets (which are inert to iodine), and the sheets are placed between the two metal plates. The plates are then compressed to the maximum capacity quickly and the pressure is released. The Teflon sheets are then removed, and the polymer film is cooled on a cold solid surface. The process may be performed as a batch process or a continuous or semi-continuous process.

The iodinated resin polymeric films described herein have a thickness ranging from about 0.01 mm to about 1.0 mm In some embodiments, the iodinated resin containing polymeric films have a thickness ranging from about 0.01 mm to about 0.3 mm. In some embodiments, the iodinated resin containing polymeric films have a thickness ranging from 0.01 mm to 0.1 mm.

As described in further detail below, the iodinated resin containing antimicrobial films of the disclosure provided herein have a high level of efficacy against a diverse array of microorganisms including various species of bacteria and fungi. In certain embodiments, the iodinated resin containing polymer films kill or deactivate at least 99% of susceptible microbes in a time period of 60 minutes or less. In particular embodiments, the iodinated resin containing antimicrobial films kill or deactivate at least 99.99% of susceptible microbes over a period of 60 minutes or less. In other embodiments, the iodinated resin containing antimicrobial films kill or deactivate at least 90% of susceptible microbes over a period of 15 minutes or less. In still other embodiments, the iodinated resin containing antimicrobial films kill or deactivate at least 99% of susceptible microbes over a period of 15 minutes or less.

It has further been found that the iodinated resin containing films of the present disclosure can kill or deactivate a significant percentage of microbes after contact times of 5 minutes or less. For instance, in some embodiments, the iodinated resin containing antimicrobial films of the present disclosure can kill or deactivate from about 20% to about 85% present of susceptible microbes after a contact time of five minutes or less. In other embodiments, the iodinated resin containing antimicrobial films of the present disclosure can kill or deactivate from about 40% to about 80% present of susceptible microbes after a contact time of five minutes or less. In still other embodiments, the iodinated resin containing antimicrobial films of the present disclosure can kill or deactivate from about 60% to about 80% present of susceptible microbes after a contact time of five minutes or less.

A significant advantage of the iodinated resin containing films of the present disclosure is there stability and their resistance to aging. Results of biological testing described below reveal that the iodinated resin containing films lose minimal or none of their total efficacy for periods of one year or more. Several of the films were subjected to an accelerated aging procedure. The iodinated resin containing films retain their high level of efficacy for at least three years.

Given the high level of efficacy, durability and lifetime of the films, they can be used in numerous healthcare related applications. For instance, it has been found that the iodinated resin containing polymeric films of the present disclosure can be used as antimicrobial surgical tapes or surgical drapes. When applied to the wound, the surgical tape or surgical drape releases iodine in a controlled manner without damaging the wound or the surrounding tissue.

The iodinated resin containing polymer films of the present disclosure are useful as surgical tapes to secure bandages or other wound dressing in place and to assist in closing wounds. In such cases, a mild adhesive will generally be applied that fastens the wound dressing to the skin. Microbes traveling in air or fluids around the wound site will be disinfected upon contact of the iodinated resin.

Occlusive or semi-occlusive surgical drapes and wound dressing components can be made using the iodinated resin films of the present disclosure. In some embodiments, a dye may be added to impart color to the wound dressing. Additionally, when applied directly to the skin, a mild adhesive may be applied to the surgical drape to assist in securing it to the wound. The adhesive may be made from a variety of polymers including but not limited to polyolefins, polyacrylates, polyesters, hydrocolloid, polyvinyl ethers and polyurethanes. An antimicrobial agent may be incorporated into the adhesive layer, thereby providing an extra layer of protection. In one embodiment, iodinated resin is incorporated into both the film and the adhesive applied to the film.

In some embodiments, the polymeric films of the present disclosure are non-adhering. Non-adhering films may optionally include a small amount of a fluoropolymer. Non-adhering films offer several advantages over adhesive tapes or dressings. When adhesive tapes or wound dressings are removed from the skin, the process is often painful. Furthermore, such skin stripping is often associated with odema and soreness. It has been found that the presently disclosed iodinated resin polymeric films are capable of forming a protective inner layer that releases iodine in a controlled manner. The inner layer is capable of ameliorating a wound by releasing iodine into the wound for a period of between about 2 days and about 14 days. In certain embodiments, non-adherent wound dressings containing an inner layer of iodinated resin are capable of releasing iodine into the wound for a period of about 7 days.

The iodinated resin containing polymer films are particularly useful for occlusive wound management. While occlusive tapes and wound dressings are designed to exclude the passage of microbes into the wound, the moisture accumulating between the wound and the interior of the film (or wound dressing) serves as a breeding ground for microbes. Although commercial products have attempted to address this issue through the addition of such agents as zinc and silver, the efficacy usually is not sufficient. Moreover, high amounts of the antimicrobial agent are often released into the wound bed, which can damage the wound and exacerbate the infection. The iodinated resin polymer films described herein provide the desired level of protection against microbes without causing damage to the wound or the surrounding tissue.

It will be further appreciated that the films of the present disclosure may be used in combination with an adsorbent material such as gauze or a nonwoven. For instance, the iodinated resin containing films may be applied as thin film backings to adsorbent materials such as pads or gauze.

In addition to wound management, the polymeric iodinated resin containing films of the present disclosure may be used in various applications. The films may also be used, for example, as (or in) packaging material; food wrap; diapers, house wrap (e.g., air/water/moisture barriers used in construction); automobile, boat, and camper covers (and other surface covers); industrial packaging; medical packaging; sports apparel and protective apparel.

### Experimental Examples

The following results show the microbiological data obtained with the iodinated resin containing polymer films of the present disclosure.

### A. Biological Testing Against Different Challenge Organisms

The following method was used to test the antimicrobial efficacy of the polymeric iodinated resin containing films of the present disclosure against different challenge microorganisms. Tests were performed using the liquid inoculum AATCC 100 Test Method (Assessment of Antibacterial Finishes on Textile Materials). In the test, circular sheets of film produced in accordance with the present disclosure Triosyn® iodinated resin films of size swatches cut into 1″×1″ film swatches and exposed to a sample of a liquid microbial suspension for contact times ranging from 5 minutes to 24 hours. The sample was then placed in a neutralizing fluid to recover viable microorganisms and the viable microorganisms were counted. Examples 1-9 show the results of various biological tests.

### Example 1

Polymer films containing 40% w/w Triosyn® T-50 powder (5-10 microns) in polypropylene were produced as described above. The polypropylene and the Triosyn® were compounded as described above. The compounded polypropylene with Triosyn® pellets were added into a circular press (0.1-0.25 g) where they are compressed while heating for approximately 10-20 seconds. The press was then re-opened and the sheet was removed and cooled on a cold surface.

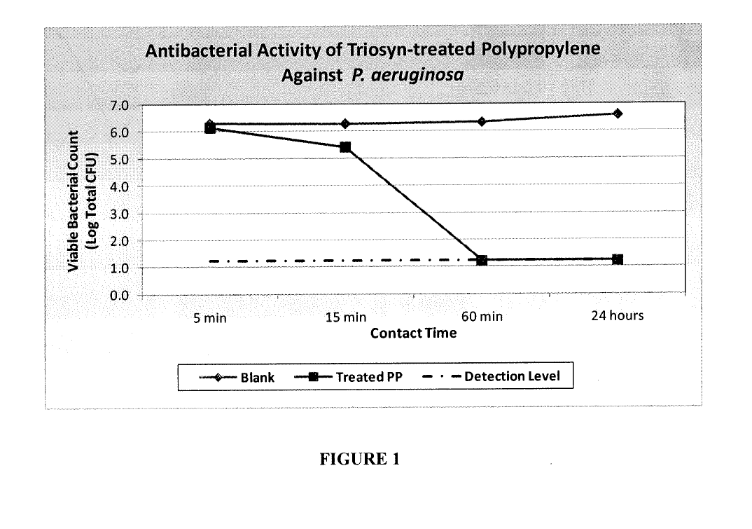

The resultant films were tested against the challenge organism Pseudomonas aeruginosa. Results from time periods ranging between 5 minutes and 24 hours are shown in Table 1 and graphically depicted in FIG. 1. The antimicrobial films show an 86.39% reduction of challenge organism after 15 minutes and a greater than 99.99% reduction after 60 minutes.

TABLE 1
Polypropylene Film Antimicrobial Performance Against
Pseudomonas aeruginosa

| Contact | Blank (n = 3) | Polyethylene + Triosyn (n = 3) | | |
|---|---|---|---|---|
| Time | (CFU Total) | (CFU Total) | Log Reduction | % Reduction |
| 5 min | 7.70E+06 | 4.70E+06 | 0.21 | 38.96% |
| 15 min | 6.05E+06 | 1.83E+06 | 0.52 | 69.70% |
| 60 min | 7.13E+06 | <1.67E+01 | >5.63 | >99.9998% |
| 24 hours | 9.00E+06 | <1.67E+01 | >5.68 | >99.9998% |

Detection Level = 16.7 CFU

### Example 2

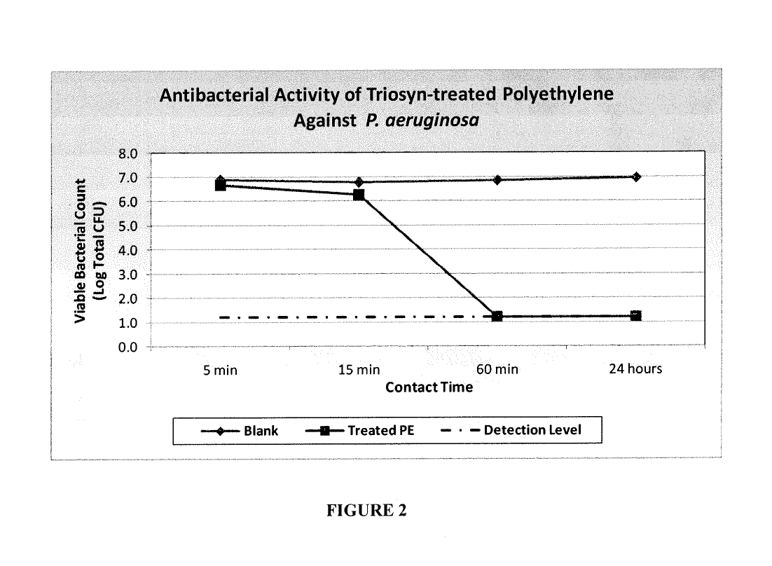

Thin films comprising 30% w/w of Triosyn® T-50 powder in polyethylene were manufactured as described in Example 1. The resultant films were tested against the challenge organism Pseudomonas aeruginosa. Results from time periods ranging between 5 minutes and 24 hours are shown in Table 2 and graphically depicted in FIG. 2. The antimicrobial films show a 69.70% reduction of challenge organism after 15 minutes and a greater than 99.99% reduction of challenge organism after 60 minutes. It is noted that in control experiments with commercially available Ioban® adhesive protective film, approximately 43% of the challenge organism Pseudomonas aeruginosa was eradicated after 60 minutes.

TABLE 2
Polyethylene Film Antimicrobial Performance Against
Pseudomonas aeruginosa

| Contact | Blank (n = 3) | Polyethylene + Triosyn (n = 3) | | |
|---|---|---|---|---|
| Time | (CFU Total) | (CFU Total) | Log Reduction | % Reduction |
| 5 min | 7.70E+06 | 4.70E+06 | 0.21 | 38.96% |
| 15 min | 6.05E+06 | 1.83E+06 | 0.52 | 69.70% |
| 60 min | 7.13E+06 | <1.67E+01 | >5.63 | >99.9998% |
| 24 hours | 9.00E+06 | <1.67E+01 | >5.68 | >99.9998% |

Detection Level = 16.7 CFU

### Example 3

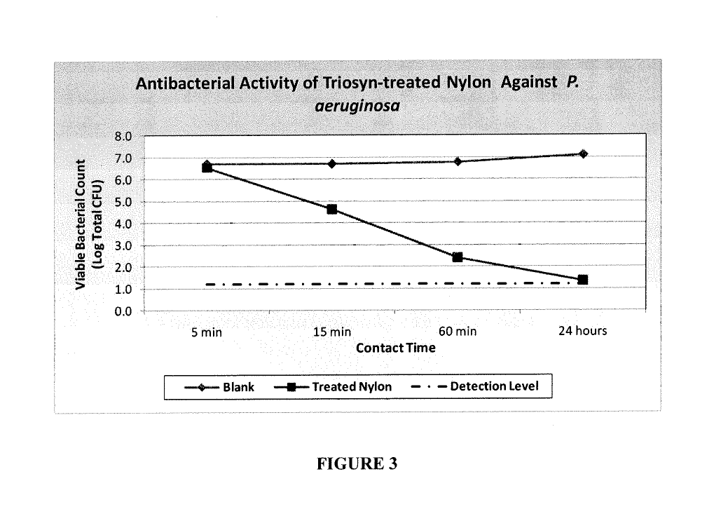

Thin films comprising 10% w/w Triosyn® T-50 powder in nylon were manufactured as described in Example 1. The resultant films were tested against the challenge organism Pseudomonas aeruginosa. Results from time periods ranging between 5 minutes and 24 hours are shown in Table 3 and graphically depicted in FIG. 3. The antimicrobial films show a 99.14% reduction of challenge organism after 15 minutes and a greater than 99.99% reduction of challenge organism after 60 minutes.

TABLE 3
Nylon Film Antimicrobial Performance Against
Pseudomonas aeruginosa

| Contact | Blank (n = 3) | Nylon + Triosyn (n = 3) | | |
|---|---|---|---|---|
| Time | (CFU Total) | (CFU Total) | Log Reduction | % Reduction |
| 5 min | 4.88E+06 | 3.43E+06 | 0.15 | 29.6928% |
| 15 min | 4.90E+06 | 4.22E+04 | 2.07 | 99.1389% |
| 60 min | 5.87E+06 | 2.61E+02 | 4.35 | 99.9955% |
| 24 hours | 1.23E+07 | 2.22E+01 | 5.74 | 99.9998% |

Detection Level = 16.7 CFU

### Example 4

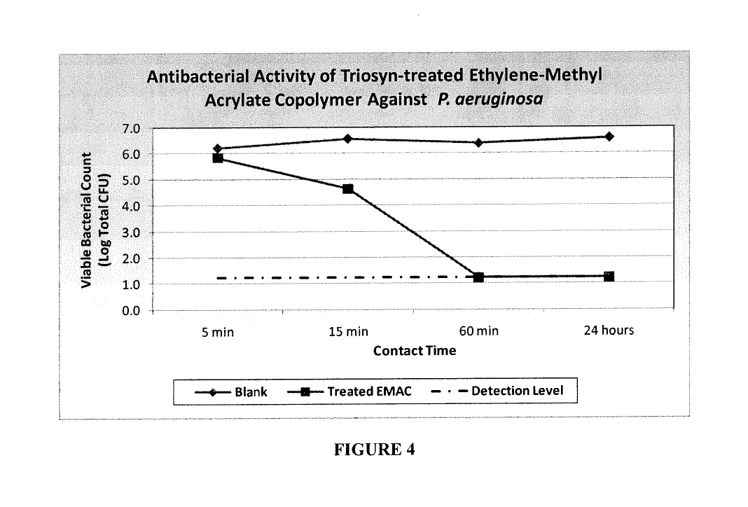

Thin films comprising 40% w/w of Triosyn® T-50 powder in EMAC were manufactured as described in Example 1. The resultant films were tested against the challenge organism Pseudomonas aeruginosa. Results from time periods ranging between 5 minutes and 24 hours are shown in Table 4 and graphically depicted in FIG. 4. The antimicrobial films show a 99.84% reduction of challenge organism after 15 minutes and a greater than 99.99% reduction of challenge organism after 60 minutes.

TABLE 4
EMAC Film Antimicrobial Performance Against
Pseudomonas aeruginosa

| | | Ethylene-Methyl Acrylate | | |
|---|---|---|---|---|
| Contact | Blank (n = 3) | Copolymer + Triosyn (n = 3) | | |
| Time | (CFU Total) | (CFU Total) | Log Reduction | % Reduction |
| 5 min | 1.63E+06 | 6.83E+05 | 0.38 | 58.16% |
| 15 min | 3.67E+06 | 4.27E+04 | 1.93 | 98.84% |
| 60 min | 2.45E+06 | <1.67E+01 | >5.15 | >99.9993% |
| 24 hours | 3.95E+06 | <1.67E+01 | >5.35 | >99.9996% |

Detection Level = 16.7 CFU

### Example 5

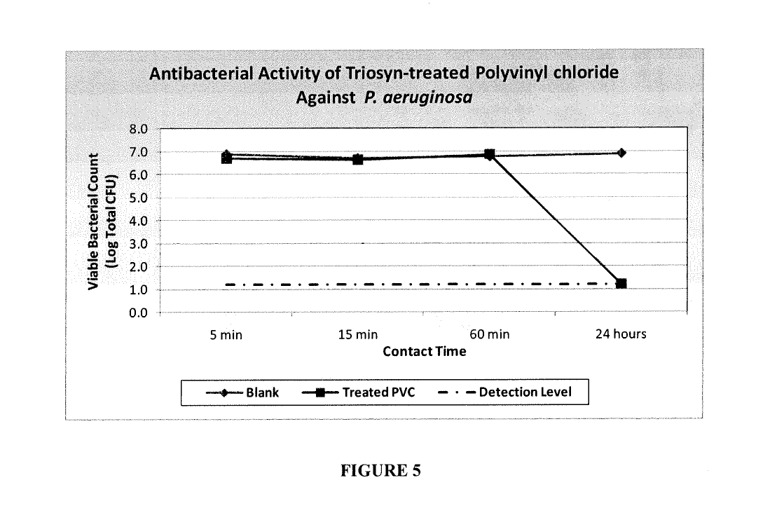

Thin films comprising 10% w/w of Triosyn® T-50 powder in polyvinyl chloride were manufactured as described in Example 1. The resultant films were tested against the challenge organism Pseudomonas aeruginosa. Results from time periods ranging between 5 minutes and 24 hours are shown in Table 5 and graphically depicted in FIG. 5. The antimicrobial films show a 26.13% reduction of challenge organism after 15 minutes and a greater than 99.99% reduction of challenge organism after 24 hours.

TABLE 5
Polyvinyl Film Antimicrobial Performance Against
Pseudomonas aeruginosa

| | | Polyvinyl chloride + Triosyn (n = 3) | | |
|---|---|---|---|---|
| Contact | Blank (n = 3) | | Log | |
| Time | (CFU Total) | (CFU Total) | Reduction | % Reduction |
| 5 min | 7.67E+06 | 4.92E+06 | 0.19 | 35.87% |
| 15 min | 4.93E+06 | 4.22E+06 | 0.07 | 26.13% |
| 60 min | 5.87E+06 | 7.30E+06 | 0.00 | 0.00% |
| 24 hours | 7.77E+06 | <1.67E+01 | >5.66 | >99.9998% |

Detection Level = 16.7 CFU

### Example 6

Thin films comprising 40% w/w of Triosyn® T-50 powder in polypropylene were manufactured as described in Example 1. The resultant films were tested against the challenge organism Pseudomonas aeruginosa either immediately after being prepared or after aging for a specific amount of time. Several samples were allowed to age for 14 days and 90 days after preparation. Other samples were subjected to accelerated aging at 45° C. for different number of days. It is noted that accelerated aging for 3 months at 45° C. corresponds to 1.5 years under real time (at 25° C., Q₁₀ factor=2.5). See ASTM International 1980-7. Results from time periods ranging between 5 minutes and 24 hours are shown in Table 6 for the various samples. The data in Table 6 reveals that aging has either no or a negligible effect on the biological performance of the antimicrobial films.

TABLE 6
Polypropylene Film Antimicrobial Performance Against Pseudomonas aeruginosa After
Aging for a Specified Period of Time

| Contact | % Reduction | | | | | |
|---|---|---|---|---|---|---|
| Time | Day 0 | Day 14 | Day 88 | Day 90 | Day 391 | Day 563 |
| 5 minutes | 32.7171% | 81.2287% | 39.3909% | | | |
| 15 minutes | 86.3894% | 76.3177% | 69.5402% | | | |
| 1 hour | >99.9992% | 99.9852% | 99.3821% | 96.6443% | 92.3232% | |
| 24 hours | >99.9996% | >99.9998% | >99.9999% | >99.8510% | >99.9990% | >99.9980% |

### Example 7

Thin films comprising 40% w/w of Triosyn® in EMAC were manufactured as described in Example 1. The resultant films were tested against the challenge organism Pseudomonas aeruginosa either immediately after being prepared or after aging for a specific amount of time. Several samples were allowed to age from 14 days to 391 days after preparation. Results from time periods ranging between 5 minutes and 24 hours are shown in Table 7 for the various samples. The data in Table 7 reveals that aging has either no or a negligible effect on the biological performance of the antimicrobial films.

TABLE 7
EMAC Film Antimicrobial Performance Against Pseudomonas aeruginosa
After Aging for a Specified Period of Time

| | % Reduction | | | | |
|---|---|---|---|---|---|
| Contact Time | Day 0 | Day 14 | Day 88 | Day 90 | Day 391 |
| 5 minutes | 58.1633% | 73.2273% | 39.1667% | | |
| 15 minutes | 98.8364% | 92.6486% | 58.6111% | | |
| 1 hour | >99.9993% | 99.9791% | 98.2081% | 99.9943% | >99.9993% |
| 24 hours | >99.9996% | 99.9998% | >99.9999% | >99.9979% | 99.9953% |

### Example 8

Thin films comprising 20% w/w of Triosyn® T-50 powder in polypropylene were manufactured as described in Example 1. The resultant films were tested against the challenge organism Staphylococcus aureus MRSA either immediately after being prepared or after aging for a specific amount of time. Several samples were allowed to age at 45° C. Results from time periods ranging between 5 minutes and 24 hours are shown in Table 8 for the various samples. The data in Table 8 reveals that aging has either no or a negligible effect on the biological performance of the antimicrobial films.

TABLE 8
Polypropylene Film Antimicrobial Performance Against Staphylococcus
aureus MRSA After Aging for a Specified Period of Time

| Contact | % Reduction | | | | |
|---|---|---|---|---|---|
| Time | Day 0 | Day 60 | Day 90 | Day 375 | Day 563 |
| 5 minutes | 99.9801% | | | | |
| 15 minutes | 92.2020% | | | | |
| 1 hours | 99.9979% | >99.998% | 99.9992% | >99.998% | >99.999% |
| 24 hours | N/A | N/A | >99.999% | N/A | >99.999% |

### Example 9

Thin films comprising 20% w/w of Triosyn® T-50 powder in polypropylene were manufactured as described in Example 1. The resultant films were tested against the challenge organism E. coli. either immediately after being prepared or after aging for a specific amount of time. Several samples were allowed to age at 45° C. Results from time periods ranging between 5 minutes and 24 hours are shown in Table 9 for the various samples. The data in Table 9 reveals that aging has either no or a negligible effect on the biological performance of the antimicrobial films.

Thin films comprising 20% w/w of Triosyn® in polypropylene were manufactured as described in Example 1. The resultant films were tested against the challenge organism E. coli. either immediately after being prepared or after aging for a specific amount of time. Several samples were allowed to age at 45° C. Results from time periods ranging between 5 minutes and 24 hours are shown in Table 9 for the various samples. The data in Table 9 reveals that aging has either no or a negligible effect on the biological performance of the antimicrobial films.

TABLE 9
EMAC Film Antimicrobial Performance Against E. coli.
After Aging for a Specified Period of Time

| Contact | % Reduction | | | | | |
|---|---|---|---|---|---|---|
| Time | Day 0 | Day 30 | Day 60 | Day 90 | Day 375 | Day 563 |
| 5 minutes | 50.9160% | 42.4460% | | | | |
| 15 minutes | 60.8443% | 79.3631% | | | | |
| 1 hours | 97.6231% | >99.9996% | | >99.999% | 99.9985% | |
| 24 hours | >99.996% | >99.9997% | >99.9997% | >99.9998% | >99.9987% | >99.9998% |

### B. Measurement of Surface Area of Iodinated Resin Particulates

### Example 10

Samples were prepared by adding 1.0-1.5 grams of powder, fragment or beads in a hollow tube and its then sealed. Samples are heated to 80° C. for 12-24 hrs to remove all traces of humidity. At this point, a known volume and pressure of nitrogen gas was passed through the sample. A multi layer of nitrogen was adsorbed at the surface of the sample until saturation. Desorption of the nitrogen is then processed and a surface area measure was then calculated. Results of the BET analysis are depicted in Table 10.

TABLE 10
BET Surface Area of Different Particle Sizes of
Triosyn ® (T50) Particulates

| Sample | BET Surface Area (m²/g) |
|---|---|
| Beads (500 micron) | 0.01165 |
| Fragments (75 micron) | 0.11 |
| Powder (5-10 micron) | 2.2092 |

Based on the BET results, powder has 190 times more surface area than beads and 20 times more than fragments of 75 micron.

1. A polymer film comprising a polymer and an iodinated resin.

2. The film of claim 1, wherein the polymer comprises a member selected from polypropylene (PP), polyethylene (PE), nylon, hydrocolloid, ethylene-methyl acrylate copolymer (EMAC), polyvinyl chloride (PVC) and combinations thereof.

3. The film of claim 1, wherein the iodinated resin is a powder.

4. The film of claim 3, wherein the surface area of the iodinated resin powder is in the range of from about 0.1. m²/g to about 3.0 m²/g.

5. The film of claim 3, wherein the surface area of the iodinated resin powder is in the range of about 2.0 m²/g to about 3.0 m²/g.

6. The polymer film of any one of claim 1, wherein the percentage of iodinated resin by weight in the film is in the range of about 5% to about 50%.

7. The polymer film of any one of claim 1, wherein the percentage of iodinated resin by weight in the film is in the range of about 10% to about 40%.

8. The polymer film of any one of claim 1, wherein said film kills or deactivates 90% or more of susceptible microbes in a period of 60 minutes.

9. The polymer film of claim 8, wherein the susceptible microbes are selected from Pseudomonas aeruginosa, E. coli, Staphylococcus aureus MRSA, and combinations thereof.

10. The polymer film of any one of claim 1, wherein said film kills or deactivates 50% or more of susceptible microbes in a period of 15 minutes.

11. The polymer film of any one of claim 1, wherein said film kills or deactivates 90% or more of susceptible microbes in a period of 15 minutes.

12. The polymer film of claim 12, wherein the susceptible microbes are selected from Pseudomonas aeruginosa, E. coli, Staphylococcus aureus MRSA, and combinations thereof.

13. A surgical tape comprising a thin film, said thin film comprising a polymer and a plurality of iodinated resin particulates incorporated in the polymer, wherein the surface area of the iodinated resin particulates is in the range of from about 1.0 m²/g to about 3.0 m²/g.

14. An occlusive wound management film, said film comprising an iodinated resin and a polymer selected from polypropylene (PP), polyethylene (PE), nylon, hydrocolloid, ethylene-methyl acrylate copolymer (EMAC), polyvinyl chloride (PVC) and an combinations thereof, wherein said film releases iodine in a controlled manner upon contact with the skin.

15. A non occlusive wound management film, said film comprising an iodinated resin and a polymer selected from polypropylene (PP), polyethylene (PE), nylon, hydrocolloid, ethylene-methyl acrylate copolymer (EMAC), and polyvinyl chloride (PVC) and combinations thereof, wherein said film releases iodine in a controlled manner upon contact with the skin.

16. The occlusive wound management film of claim 14, further comprising a dye.

17. The occlusive wound management film of claim 15, further comprising a dye.

18. The wound management film of any one of claim 14, further comprising an adhesive.

19. A non-adherent thin film, said film comprising a polymer and an iodinated resin.

20. The non-adherent thin film of claim 19, wherein the polymer is selected from the group consisting of polypropylene (PP), polyethylene (PE), nylon, hydrocolloid, ethylene-methyl acrylate copolymer (EMAC), and polyvinyl chloride (PVC).

21. The non-adherent thin film of claim 19, wherein surface area of the iodinated resin on the polymer is in the range of from about 1.0 m²/g to about 3.0 m²/g.

22. A method for manufacturing an iodinated polymer film, the method comprising the steps of:
i. introducing an iodinated resin powder into an extruder;
ii. introducing polymer into the extruder;
iii. operating the extruder at a temperature such that the polymer is molten;
iv. cooling the polymer/iodinated resin extrudate to at least partially solidify the extrudate;
v. cutting the extrudate into small pieces;
vi. sandwiching the small pieces of extrudate between sheets;
vii. compressing the sheets at high temperature and pressure;
viii. removing the polymer film formed between the sheets;
ix. and cooling the polymer film.

23. The method of claim 22, wherein the cutting step comprises pelletizing the polymer/iodinated resin extrudate.

24. The method of claim 22, wherein the polymer comprises at least one member selected from the group consisting of polypropylene (PP), polyethylene (PE), nylon, ethylene-methyl acrylate copolymer (EMAC), and polyvinyl chloride (PVC).

25. The method of claim 22, wherein the extruder is a twin-screw extruder.

26. The method of claim 22, wherein the extruder is coated or lined to improve corrosion resistance.
